(19) United States
(12) Patent Application Publication
(10) Pub. No.: US 2017/0016892 A1
(43) Pub. Date: Jan. 19, 2017

(54) LOCALIZED SURFACE PLASMON RESONANCE SENSING OF HUMAN PERFORMANCE BIOMARKERS USING SHORT PEPTIDE RECOGNITION ELEMENTS ON OPTICALLY ACTIVE METAL NANOSTRUCTURES

(71) Applicant: Government of the United States as Represented by the Secretary of the Air Force, Wright-Patterson AFB, OH (US)

(72) Inventors: Rajesh R. Naik, Centerville, OH (US); Joshua A. Hagen, Cincinnati, OH (US); Joshua M. Slocik, Dayton, OH (US); Abrin L. Schmucker, Midland, MI (US); Srikanth Singamaneni, St. Louis, MO (US)

(73) Assignee: Government of the United States as Represented by the Secretary of the Air Force, Wright-Patterson AFB, OH (US)

(21) Appl. No.: 15/210,398

(22) Filed: Jul. 14, 2016

Related U.S. Application Data
(60) Provisional application No. 62/192,152, filed on Jul. 14, 2015.

(51) Int. Cl. G01N 33/543; G01N 33/558; G01N 21/552; G01N 33/553

(52) U.S. Cl. CPC G01N 33/54373; G01N 33/54346; G01N 33/553; G01N 33/558; G01N 21/554; G01N 2201/06113

(57) ABSTRACT

A biomarker sensor using short peptide recognition elements. The biomarker sensor includes a substrate and a metallic layer on a surface of that substrate that is localized surface plasmon resonance reactive. A receptor layer on the surface of the metallic layer (the surface opposing the substrate) is configured to selectively bind a biomarker and has a thickness less than about 15 nm.

Pursuant to 37 C.F.R. §1.78(a)(4), this application claims the benefit of and priority to prior filed co-pending Provisional Application Ser. No. 62/192,152, filed Jul. 14, 2015, which is expressly incorporated herein by reference in its entirety.

### RIGHTS OF THE GOVERNMENT

The invention described herein may be manufactured and used by or for the Government of the United States for all governmental purposes without the payment of any royalty.

### FIELD OF THE INVENTION

The present invention relates generally to biosensors and, more particularly, to localized surface plasmon resonance based biosensors.

### BACKGROUND OF THE INVENTION

Biosensors based on localized surface plasmon resonance (“LSPR”) are highly attractive for lab-on-chip devices because of their cost-effectiveness and availability for point-of-care biodiagnostics. Metal nanostructures used in LSPR have been shown to be sensitive enough to differentiate various inert gases (refractive index differences on the order of 3×10⁻⁴), probe the conformational changes of individual biomacromolecules, detect single biomolecule binding events, monitor the kinetics of catalytic activity of single nanoparticles, and optical detection of a single electron. Two factors are of prime importance in designing LSPR-based biosensors: (1) a bulk refractive index sensitivity, and (2) electromagnetic decay length of the metal nanostructures employed.

Specific biomolecular interactions, such as antibody-antigen interactions, form the basis for numerous bioassays, including enzyme-linked immunosorbent assay (“ELISA”), immunoblotting (or western blotting), and immunoprecipitation. Conventionally, plasmonic biosensors, such as the sensor 50 illustrated in FIG. 1, include a substrate 52, a metal nanostructure 54, with antibodies 56 as recognition elements for particular targets 58. While antibody-based biosensors 50 offer excellent molecular recognition capabilities, there remain some drawbacks. Particularly, antibody biosensors 50 are deficient in limited pH and temperature stability, loss of conformation and recognition functionality in non-aqueous media, high cost associated with generating antibodies, and poor compatibility with micro and nanofabrication processes for efficient integration with various transduction platforms.

Owing to the evanescent nature of electromagnetic fields at surfaces of the metal nanostructures, the LSPR wavelength shift exhibits a characteristic decay with increasing distance, which is given by:

$$R = m Δη ( 1 - exp ( - \frac{d}{{l}_{d}} ) ) \qquad Equation 1$$

where R is the LSPR shift, m is a bulk refractive index sensitivity (“RIS”), Δη is a difference in refractive index between an adsorbed layer and a surrounding medium, d is a layer thickness, and l_{d} is an electromagnetic decay length. Therefore, the LSPR shift measured at a binding event depends on the RIS and decay length, which are characteristic to a given biosensor. The large size of natural antibodies 56 (often on the order of about 150 KDa and illustrated in FIG. 1 as “l₁”) inherently and significantly decreases the sensitivity of LSPR-based biosensors as compared to SPR-based sensors.

It would be of great benefit to develop a universal sensing platform, which leverages the optical properties of plasmonic nanoparticles functionalized with peptide-based recognition elements, for the detection of human performance markers in biological fluids at physiologically relevant levels.

### SUMMARY OF THE INVENTION

The present invention overcomes the foregoing problems and other shortcomings, drawbacks, and challenges of antibody-based biosensors. While the invention will be described in connection with certain embodiments, it will be understood that the invention is not limited to these embodiments. To the contrary, this invention includes all alternatives, modifications, and equivalents as may be included within the spirit and scope of the present invention.

One embodiment of the present invention is directed to a biomarker sensor having a substrate and a metallic layer on a surface of that substrate that is localized surface plasmon resonance reactive. A receptor layer on the surface of the metallic layer (the surface opposing the substrate) is configured to selectively bind a biomarker and has a thickness less than about 15 nm.

According to an embodiment of the present invention, a biomarker sensor comprises a substrate and metallic nanoparticles configured to adhere to that substrate. The metallic nanoparticles are localized surface plasmon resonance reactive. A plurality of peptides is coupled to each metallic nanoparticle. Each peptide of the plurality is selective to a desired biomarker and has a number of amino acids ranging from about 12 to about 65.

Still another embodiment of the present invention includes a biomarker sensing device comprising a support with the biomarker sensor supported on the support. At least one flow path extends across the support and crosses the biomarker sensor.

Yet other embodiments of the present invention are directed to a biomarker sensor having a substrate, a metallic layer that is localized surface plasmon resonance reactive, and a receptor layer. The metallic layer is on a surface of the substrate and the receptor layer is on a surface of the metallic layer that is opposite the substrate. The receptor layer has a thickness of about 15 nm.

In accordance with other embodiments of the present invention, a biomarker sensing device includes a substrate having a surface, first and second biomarker sensors, and a flow path. Each of the first and second biomarker includes a respective metallic layer on the surface of the substrate and a respective receptor layer on a surface of associated metallic layer that is localized surface plasmon resonance reactive. The first receptor layer is configured to selectively bind a first biomarker; the second receptor layer is configured to selectively not bind the first biomarker. First and second portions of the flow path are configured to intersect with the first and second biomarker sensors, respectively.

According to still other embodiments of the present invention, a biomarker sensor system includes a substrate having a metallic layer on a surface of the substrate that is localized surface plasmon resonance reactive and a receptor layer on a surface of the metallic layer opposing the substrate. The receptor layer is configured to selectively bind a biomarker. A fluid path intersects the receptor layer and is configured to flow a specimen across the substrate and the receptor layer. A detector measures a physical character of the metallic layer such that the physical character changes when the specimen includes the desired biomarker and the desired biomarker binds to the receptor layer and the physical character does not change when the specimen does not include the biomarker and none of the desired biomarker binds to the receptor layer.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a cross-sectional view of a cross-sectional view of a localized surface plasmon resonance sensor according to the prior art.

FIG. 2 is a cross-sectional view of a cross-sectional view of a localized surface plasmon resonance sensor according to one embodiment of the present invention.

FIG. 2A is a cross-sectional view of a localized surface plasmon resonance sensor according to yet another embodiment of the present invention.

FIG. 3 is a flowchart illustrating a method of preparing a localized surface plasmon resonance sensor according to an embodiment of the present invention.

FIG. 4 is a schematic and sequential illustration of the method of FIG. 3.

FIG. 5 is a perspective view of a localized surface plasmon resonance sensor according to another embodiment of the present invention.

FIG. 6 is a side elevational view of a localized surface plasmon resonance sensor coupled to a wicking substrate according to an embodiment of the present invention.

FIG. 7 is a cross-sectional view taken along the line 7-7 of FIG. 6.

FIG. 8 is a top down view of an analytical device incorporating localized surface plasmon resonance sensors according to embodiments of the present invention.

FIG. 9 is an image of gold nanorods synthesized using a seed-mediated appropriate and having an average length of 47.3±2.3 nm and an average diameter of 20.2±1.4 nm.

FIG. 10 is a graphical representation of a red-shift in the LSPR spectrum after a peptide binds to the gold nanorods of FIG. 9.

FIG. 11 is a graphical representation of a red shift in the LSPR spectrum after an antibody binds to the gold nanorods of FIG. 9.

FIG. 12 is a scanning electron microscope image of peptide- and antibody-conjugated gold nanoparticles absorbed onto a filter paper substrate.

FIGS. 13A and 13B are graphical representations of red-shifts in the LSPR spectrum of gold nanorods having bound peptide and antibody, respectively.

FIG. 14 is a graphical representation of

FIG. 15 is a graphical representation of the differences in red shift in the LSPR spectra of gold nanorods having bound peptide as compared to bound antibody.

FIGS. 16A and 16B are graphical representations measured LSPR shift exhibited a monotonic increase with increased troponin concentration in both human plasma and artificial eccrine sweat, respectively.

FIG. 17 is a graphical representation of the evolution of a thickness of a peptide layer and a peptide-cTnI layer on a gold surface.

FIG. 18 is a graphical representation of a distance-dependent refractive index sensitivity parameter related to the LSPR shift.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF THE INVENTION

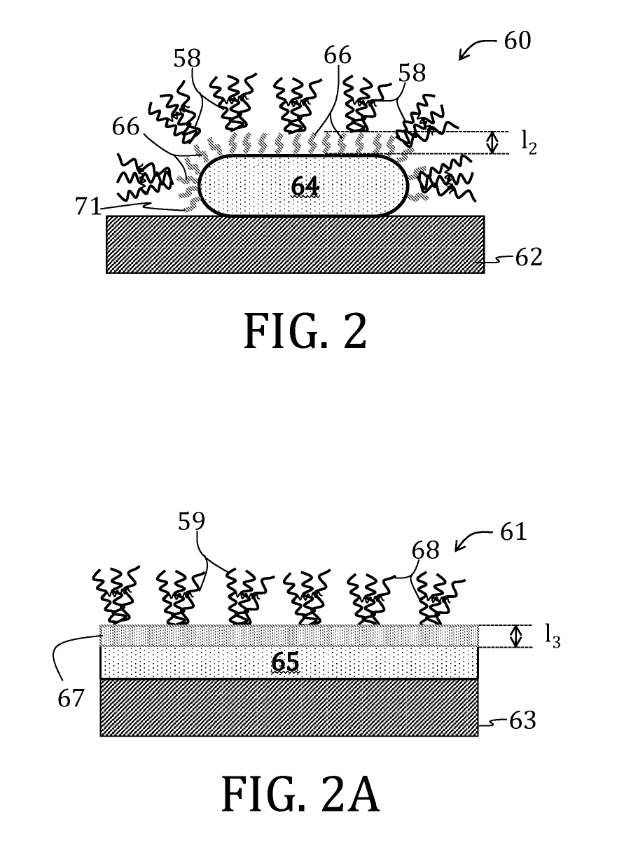

Turning now to the figures, and in particular to FIG. 2, a localized surface plasmon resonance (“LSPR”) based optical sensing platform (hereafter, “the sensor” 60) according to an embodiment of the present invention is shown. A second embodiment of the invention is illustrated in FIG. 2A and described in greater detail below.

The sensor 60 generally includes a substrate 62 having a metallic layer, specifically illustrated here as nanostructures 64 functionalized with a receptor layer, specifically here, peptides 66. The substrate 62 may comprise one of various structures, including, for example, glass, filter paper, or polymers, such as Polyethylene terephthalate (PET), KAPTON (E. I. du Pont de Nemours and Co., Wilmington, Del.) or polyimide. Generally, the substrate 62 need only be a material suitable for use in LSPR detection techniques and for supporting the metallic nanostructures 64.

The metallic nanostructures 64 may be gold nanorods (hereafter, “AuNRs”) or gold nanocubes. As would be known by those skilled in the art having the benefit of the disclosure herein, the shape, size, and composition, may all be used to tune and maximize the sensor's LSPR response. In any event, the metallic nanostructures 64 may be synthesized (such as synthesis using a seed-mediated approach) or otherwise purchased for use from suitable vendors, for example.

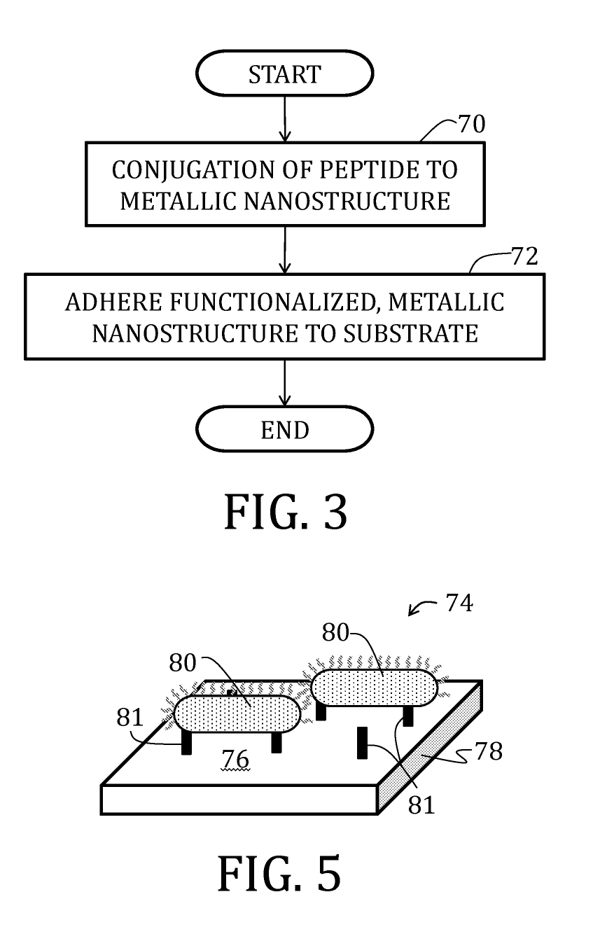

Selection of the peptides 66 is largely dependent on the desired target and level of specificity. The peptides may be selected from one or more peptide libraries or can be rationally designed using molecular modeling. For purposes of illustration with reference to FIGS. 2 and 3, the selected peptide is troponin I binding peptide and comprises Seq. ID No. 1.

According to the embodiment of FIG. 2A, the sensor 61 generally includes a substrate 63 having a metallic layer thereon 65 with receptor layer 67. For the embodiment of the invention illustrated in FIG. 2A, as compared to the sensor 60 of FIG. 2, the metallic layer 65 may be configured so as to provide an LSPR response. Deposition methods may include, but are not limited to nanosphere lithography and atomic layer deposition, sputter coating, and metal evaporation for use with silver, gold, copper, platinum, palladium, and aluminum. The receptor layer 67 comprises, at least in part, peptides as described previously. The receptor layer 67 may, therefore include a polymer having the peptides bound thereto. One exemplary peptide conjugated polymer would be a troponin specific binding peptide conjugated to a polyethylene glycol, which may vary in length.

The substrate 62 may comprise one of various structures, including, for example, glass, filter paper, or polymers, such as Polyethylene terephthalate (PET), KAPTON (E. I. du Pont de Nemours and Co., Wilmington, Del.) or polyimide. Generally, the substrate 62 need only be a material suitable for use in LSPR detection techniques and for supporting the metallic nanostructures 64.

The metallic nanostructures 64 may be gold nanorods (hereafter, “AuNRs”) or gold nanocubes. As would be known by those skilled in the art having the benefit of the disclosure herein, the shape, size, and composition, may all be used to tune and maximize the sensor's LSPR response. In any event, the metallic nanostructures 64 may be synthesized (such as synthesis using a seed-mediated approach) or otherwise purchased for use from suitable vendors, for example.

Selection of the peptides 66 is largely dependent on the desired target and level of specificity. The peptides may be selected from one or more peptide libraries or can be rationally designed using molecular modeling. For purposes of illustration with reference to FIGS. 2 and 3, the selected peptide is troponin I binding peptide and comprises Seq. ID No. 1.

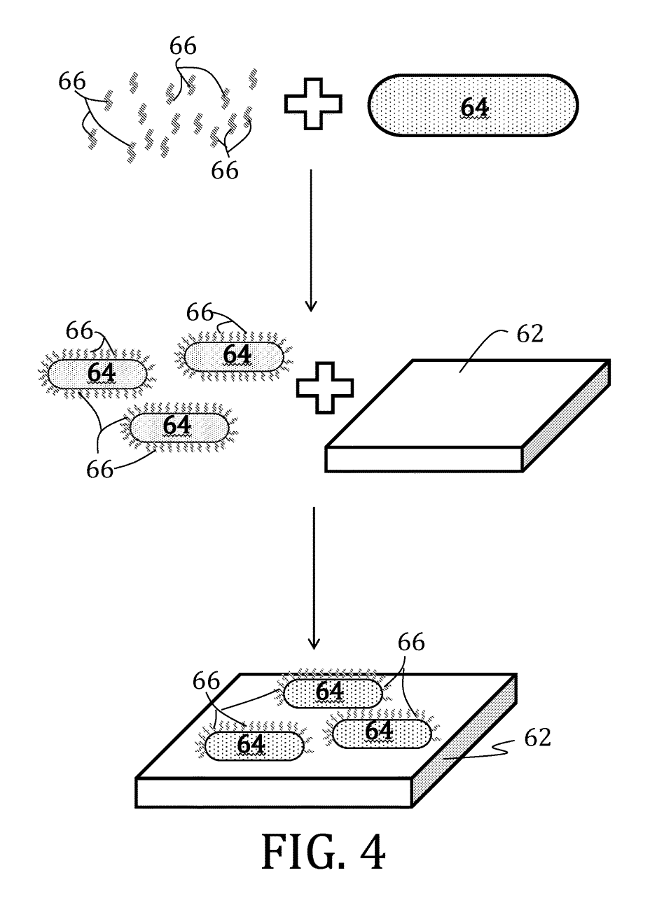

Referring now to FIGS. 3 and 4, with continued reference to FIG. 2, and with suitable materials for the sensor 60 selected according to the above teachings, a method of preparing sensors 60 according to an embodiment of the present invention is described. In Block 70, the selected peptide 66 may be conjugated to the metallic nanostructure 64. For example, and in accordance with the illustrative embodiment, the troponin I binding peptide was conjugated to the AuNPs 64 by introducing a cysteine residue at the C-terminal, comprising Seq. ID No. 2, so as to facilitate binding through an Au—S linkage.

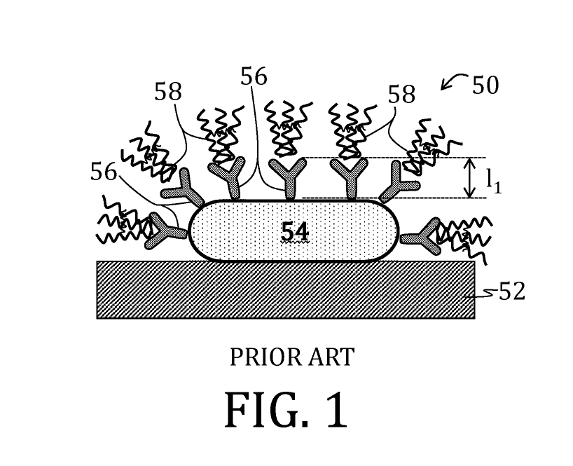

The smaller size of the peptide 66 as compared to the large antibody 56 (FIG. 1) (illustrated in FIG. 2 as “l₂”) inherently and significantly increases the sensitivity of LSPR-based biosensors, again, as compared to the conventional, antibody counterparts. Generally, the peptide 66 will have a number of amino acids ranging from about 12 to about 65. According to some embodiments, the peptide 66 has a length of about 15 nm or such that when the peptide 66 is conjugated to the metallic nanostructure 64, a distal end 71 of each peptide 66 is no more than about 15 nm away from the metallic nanostructure 64.

The now functionalized, metallic nanostructures 64 may then be adsorbed or otherwise adhered to the substrate 62 (Block 72). According to some embodiments of the present invention, such as the sensor 74 illustrated in FIG. 5, a surface 76 of the substrate 78 may be previously functionalized so as to immobilize the functionalized, metallic nanostructures 64. Functionalization may include mercaptosilane 81, which reduces aggregation, surface diffusion, or both when the substrate 78 is exposed to high ionic strength environments (such as perspiration).

Returning again to FIG. 4, and according to other embodiments of the present invention, the substrate 62 may be porous, wicking, or both, such as filter paper. Therein, the functionalized, metallic nanostructures 64 are absorbed by immersing the filter paper 62 into a solution of the functionalized, metallic nanostructures (solution not shown), which becomes saturated with the solution.

With adhesion complete, the sensor 60 may be used for detection presence of targets, which is described in greater detail below.

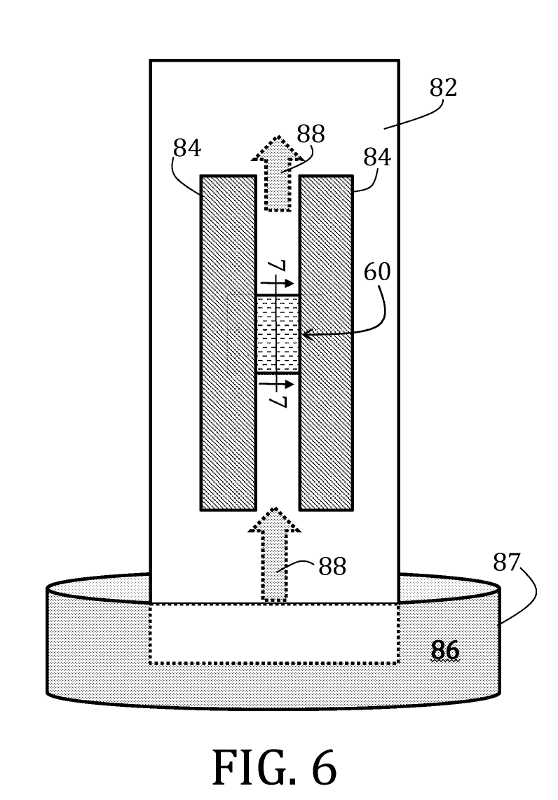

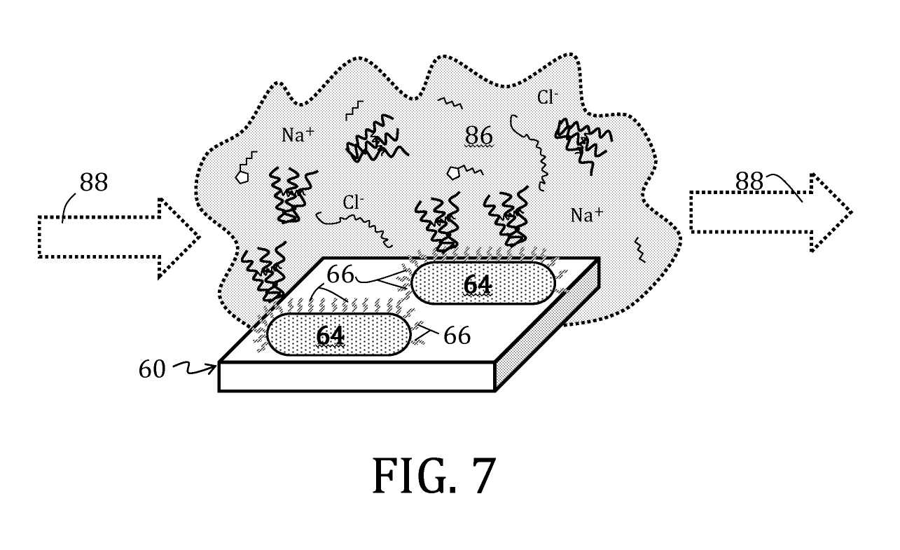

FIGS. 6 and 7 illustrate use of the sensor according to an embodiment of the present invention. Generally, the sensor 60 secured via one or more securement structures 84 to a larger, wicking substrate 82, which may include a glass slide, filter paper, or polymers, such as PET, KAPTON or polyimide. A fluid specimen 86 may flow across the sensor 60, such as in the direction of arrows 88. The fluid specimen may comprise one or more of any number of fluids, including biological fluids (such as, sweat, blood, urine, and so forth) or nonbiological fluids (such as, solutions prepared in a laboratory for purposes of testing).

In FIG. 7, which is a cross-sectional view of the sensor 60 taken along the line 7-7 in FIG. 6, illustrates the flowing of liquid specimen 86 (shown in FIG. 7 as sweat) over the sensor 60, wherein molecules of the target 58 may be captured and adhere the peptides 66. While flowing, the interactions between peptides 66 and target 58, if present, may be characterized using surface plasmon resonance, as would be understood by those skilled in the art. Non-binding elements (for example, salts, short chain molecules, etc.) within the fluid specimen 86 pass by the sensor 60 without binding. Briefly, the larger substrate 82 and fluid sample supply 87, with associated flow channel hardware (pumps, tubing, and so forth, not shown) are prepared with respect to a surface plasmon resonance spectrometer (not shown). The spectrometer includes a light source (for example, a laser) configured to direct light toward the larger substrate 82 with the sensor 60. The light enters a prism adjacent fluid specimen flowing over the sensor where the light undergoes total internal reflection until being reflected out of the prism toward a detector. The reflected light is acquired at the detector and an attenuation of the reflected light as compared to the initial, incident light is determined. The amount of attenuation may then be related to binding of target to the peptide.

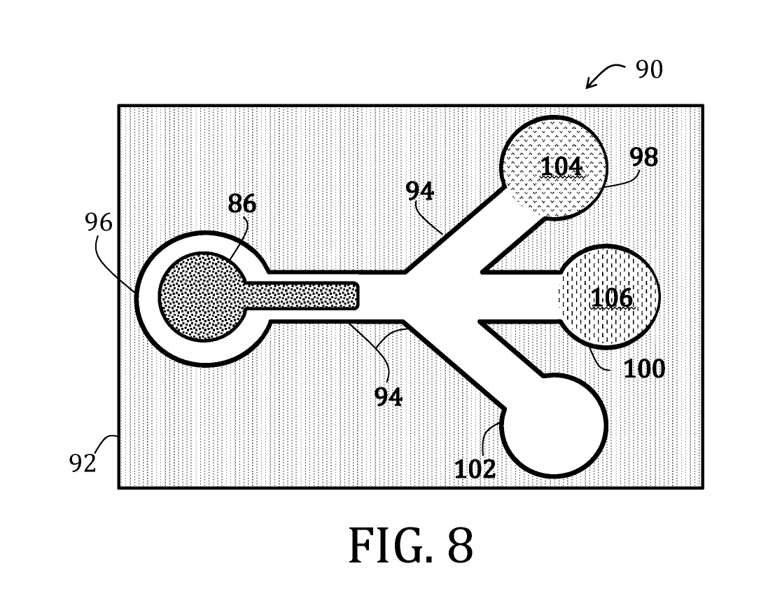

Referring now to FIG. 8, an analytical device 90 according to another embodiment of the present invention is shown. The device 90 includes a substrate 92, which may be constructed on a wicking material, for example, filter paper having graduated porosity. A plurality of fluid channels 94 are formed on the surface of the substrate 92 (such as by wax inject printing or other patterning technique), the number of depending, at least partially, on a number of targets to be analyzed, a number of negative controls desired, a number of positive controls desired, and so forth. As shown in FIG. 8, the plurality of fluid channels 94 are fluidically coupled to a specimen well 96, a test well 98, a negative control well 100, and a blank well 102, such that the number of fluid channels 94 comprising the plurality is four.

The specimen well 96 is configured to receive the fluid specimen 86 and to enable movement of the fluid specimen 86 from the specimen well 96, along the plurality of fluid channels 94, to the test, negative control, and blank wells 98, 100, 102. The test well 98 may have a functional peptide nanoparticle surface 104 in which the surface of the substrate 92 within the test well 98 is coated with metallic nanostructures 64 (FIG. 2) functionalized with peptide 66 (FIG. 2) configured to bind a desired target 58 (FIG. 2). The negative control well 100 may have a non-functional peptide nanoparticle surface 106 in which the surface of the substrate 92 within the negative control well 100 is coated with metallic nanostructures having non-specific peptides or otherwise benign peptides that generally do not bind the desired target 58 (FIG. 2). The blank well 102 may be devoid of coating or nanoparticles.

In use, the device 90 may be set up with respect to surface plasmon resonance spectrometer. Irradiation of the device 90 occurs while fluid specimen 86 flows from the specimen well 96 and though the plurality of fluid channels 94 to the test, negative control, and blank wells 98, 100, 102. If the target 58 (FIG. 2) is present, then binding between the target 58 (FIG. 2) and the functional peptide nanoparticles comprising the associated surface 104 may result in attenuation of reflected light associated with the functional peptide nanoparticle surface 104. Specificity of binding may be evaluated by evaluating attenuation of reflected light associated with the negative control well 100 and with the blank well 102.

While illustrative and demonstrative embodiments are described herein relative to troponin I binding peptide, it would be understood by the skilled artisan having the benefit of the disclosure provided herein that other peptides and targets may be selected. For instance, neuropeptide Y (comprising Seq. ID No. 3) having a modified c-terminal cysteine residue (comprising Seq. ID No. 4) for detection of Neuropeptide Y, and peptides selective to cytokines like Interleukin 6, Interleukin 10, tumor necrosis factor, and cortisol, dopamine, epinephrine, viruses, antibodies, bacteria, chemical agents may be used. However, this listing should not be considered to be fully encompassing of all possible embodiments. The pairing of peptides, known for their small sensing volumes and high target-binding affinities, with the sensitivity offered by anisotropic nanoparticles in LSPR-based sensors, offers much greater sensitivity than when paired with larger bulkier capture agents (i.e., antibodies). Importantly, once standard curves for individual targets have been generated, this method enables both detection and quantification of target for a given sample.

The following examples illustrate particular properties and advantages of some of the embodiments of the present invention. Furthermore, these are examples of reduction to practice of the present invention and confirmation that the principles described in the present invention are therefore valid but should not be construed as in any way limiting the scope of the invention.

### Example 1

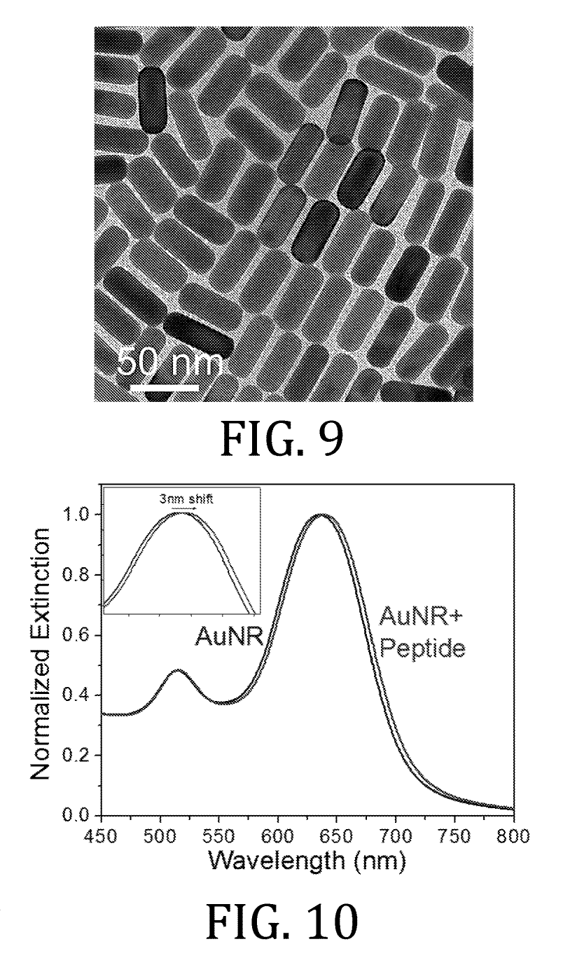

AuNRs were synthesized using a seed-mediated approach with an average length of 47.3±2.3 nm and an average diameter of 20.2±1.4 nm (see FIG. 9).

Human troponin I binding peptide (Seq. ID No. 1), identified from a phage display peptide library, with nanomolar (nM) binding affinity to cTnI. Conjugation of the peptide to AuNR was achieved by introducing a cysteine resident at the C-terminus of the peptide (Seq. ID No. 2), to facilitate binding via an Au—S linkage.

While not wishing to be bound by theory, it is believed that the structure-based functional mechanism of the peptide-conjugated BPDs. Using molecular dynamic (“MD”) simulations, the structure and thickness layer of the peptide recognition element on the gold surface was investigated. The atomistic structure of the cTnI binding peptide showed N-terminal Phe1 and C-terminal Cys13 residues in close contact with the gold surface. The amino acid Trp10 in the peptide (possible binding residue to cTnI) was distal to the gold surface and accessible for cTnI binding. Three amino acid residues within the binding pocket, Lys58, Glu6, and Arg 136, are available for non-covalent interaction with cTnI binding peptide on the surface of the gold.

As shown in FIG. 10, and at binding of the peptide to the AuNR surface, a LSPR wavelength red-shift of about 3 nm was observed and attributed to an increase in the refractive index of the medium surrounding the AuNRs.

Peptide conjugated AuNRs were adsorbed on the paper substrate and confirmed using surface enhanced Raman scattering (“SERS”) spectroscopy. SERS spectra revealed Raman bands corresponding to C—C and C—N⁺ vibrations at 1004 cm⁻1 from phenylalanine and 1341 cm⁻¹ and 1360 cm⁻¹ from tryptophan.

### Example 2

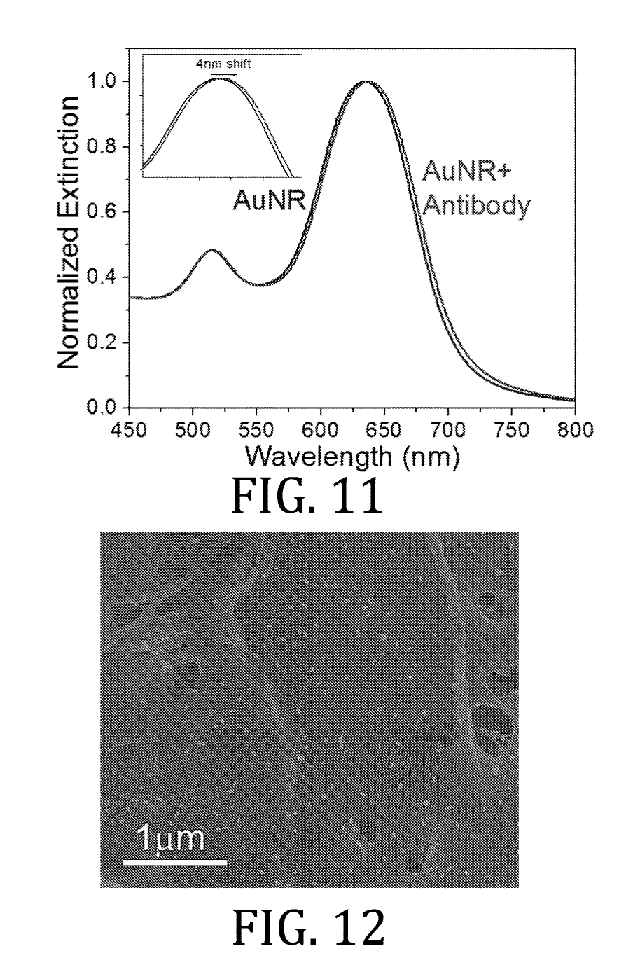

Performance of peptide paper substrates prepared in accordance with Example 1 was compared with performance of conventional antibody BPDs. As such, anti-cTnI (N-terminus), a polyclonal antibody, was used as an antibody recognition element. Anti-cTnI was conjugated to the AuNRs using a carbodiimide cross linker chemistry and thiol-terminated poly (ethylene glycol) (“SH-PEG”). Confirmation of the antibody's affinity to troponin after bioconjugation with SH-PEG was made by dot-blot dilution immunoassay. The pegylated anti-cTnI was found to bind to the surface of the nanotransucer via an Au—S linkage. As shown in FIG. 11, coupling of anti-cTnI to AuNRs resulted in a red shift in the LSPR wavelength of about 4 nm.

Dynamic light scattering (“DLS”) was used to monitor changes in the hydrodynamic size of the AuNR at bioconjugation with cTnI binding peptide or anti-cTnI. The increase in the hydrodynamic radius of the peptide-conjugated AuNR was smaller than the increase observed for the antibody-conjugated AuNR (1.4 nm versus 11.8 nm, respectively), which was attributed to the relative molecular weights (1.6 kDa versus 150 kDa, respectively).

A dry state thickness of the peptide and antibody recognition layers was measured using atomic force microscopy (“AFM”). The results indicated that an increase in diameter of the AuNRs after antibody conjugation (about 4.2 nm), which was significantly higher compared to that after peptide conjugation in the dried state (diameter being about 1 nm).

### Example 3

Peptide- and antibody-conjugated AuNPs, prepared in accordance with embodiments taught herein, were adsorbed on a filter paper in accordance with embodiments described herein. Briefly, a 1×1 cm strip of filter paper (such as Whatman #1) was immersed in a solution until saturation of peptide-conjugated AuNR.

FIG. 12 is an exemplary scanning electron microscope (“SEM”) image of the resultant paper, which revealed a uniform distribution of the bioconjugated AuNRs with no signs of aggregation or patchiness. Areal densities of the AuNR-antibody and AuNR-peptide conjugates adsorbed on the paper surface were found to be 52±3 μm⁻² and 49±2 μm⁻², respectively.

Density of AuNRs was quite similar in both cases, minimizing effects on sensitivity due to variations in density. Extinction spectra were collected from several spots (about 2 μm² areas, corresponding to about 200 nanorods) across the surface of the paper were collected using a microspectrometer mounted on an optical microscope and showed excellent optical uniformity with a standard deviation of less than 1 nm in longitudinal wavelength. For each, baseline was subtracted and was then deconvoluted using a two peak Gaussian fit.

The concentration of the AuNR solution used for immersion was critical to achieve an optically homogeneous sensing substrate, which, in turn, is critical in the design of a biosensor. Optical uniformity defines the noise level of a biosensor and determines the limit of detection (“LOD”). Longitudinal LSPR was used for detecting and monitoring target binding events and, after absorption, LSPR wavelength of AuNR exhibited a blue shift of about 17 nm as compared to the same in solution. This blue shift was believed to be due to a decrease in the effective refractive index of the surrounding medium from water-to-air and paper substrate.

### Example 4

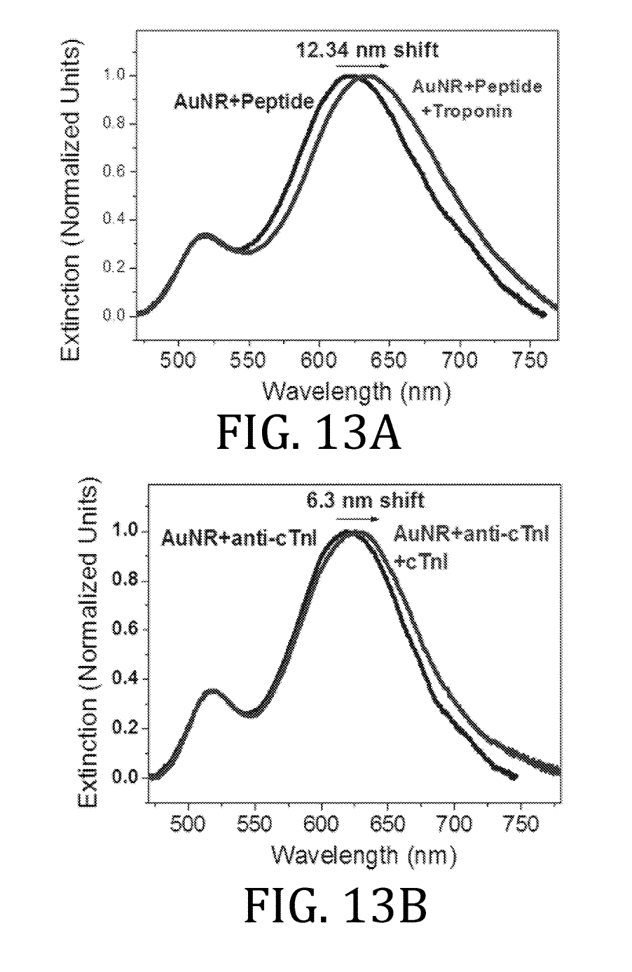

To probe the sensing capability of the BPDs, each BPD was exposed to troponin 1 (3.53 μg/mL in 0.1 M TBS, pH 8.0). Extinction coefficient obtained from the peptide-AuNR and antibody (anti-cTnI)-AuNR BPDs showed a red shift of 12.3 nm and 6.3 nm, respectively, in FIGS. 13A and 13B. The higher sensitivity of peptide-AuNR as compared to the sensitivity of the antibody-AuNR was believed to be due to differences in the thickness of the peptide and antibody adsorbate layer.

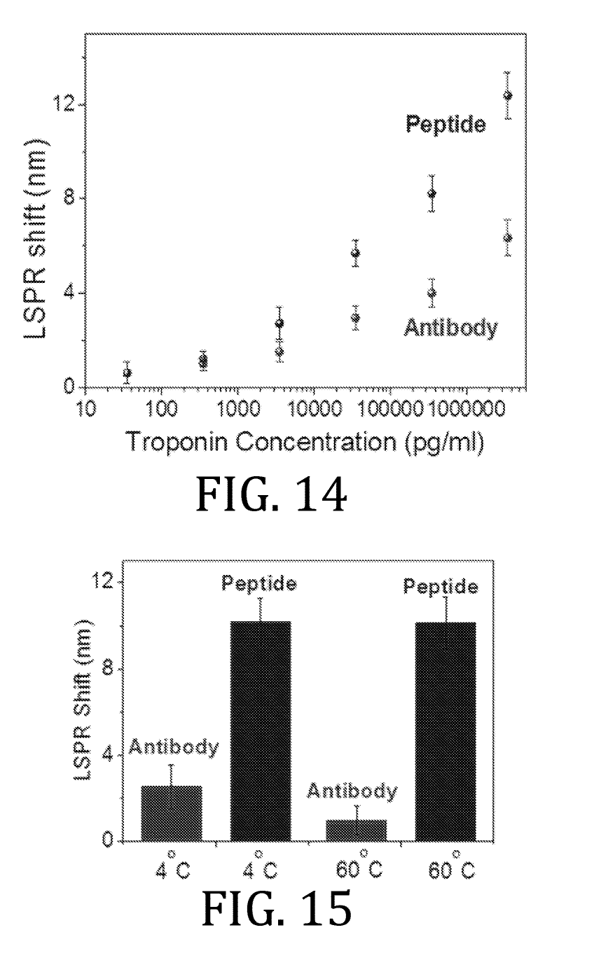

To probe the limit of detection in both the peptide- and antibody-AuNR BPDs, each BPD was exposed to varying concentrations of cTnI. In both BPDs, a monotonic increase in the LSPR shift was observed with increasing concentration of cTnI (see FIG. 14). The limit of detection for peptide-based BPDs was found to be about 35.3 pg/mL, which was an order of magnitude lower than the sensitivity of the antibody-based BPDs (about 353 pg/mL).

To compare selectivity of both the peptide- and antibody-AuNR BPDs towards cTnI, human serum albumin (“HSA”) was used as an interfering protein. Both antibody and peptide-modified AuNR exhibited small LSPR shifts upon exposure to high concentrations of HSA demonstrating excellent selectivity of the bioconjugated AuNR to cTnI.

To compare stability of both the peptide- and antibody-AuNR BPDs, each BPD type was incubated at 4° C. and at 60° C., each for 48 hrs. The peptide-based BPD exhibited remarkable thermal and temporal stability via consistent, measured LSPR shift. However, antibody-based BPD demonstrated degradation of performance via smaller LSPR shift after exposure. This reduction is believed to be due to antibody denaturation and lose molecular recognition at elevated temperatures. Moreover, antibody-AuNR BPDs stored at 4° C. for 48 hours demonstrated reduced LSPR shift as compared to freshly prepared antibody-AuNR BPDs, which was believed to be due to poor shelf-life stability of antibodies in a dry state (FIG. 15). The demonstrated shelf-life stability makes peptide-based BREs excellent candidates for LSPR-based point-of-care diagnostics in resource-limited settings.

### Example 5

In view of the high sensitivity and shelf-life stability of peptide-based BPDs, the detection of troponin in complex physiological fluids, such as human plasma and sweat, was considered. Cardiac troponins in blood are considered to be the most sensitive and specific biomarkers for myocardial infarction, a medical condition that involves myocardial tissue injury.

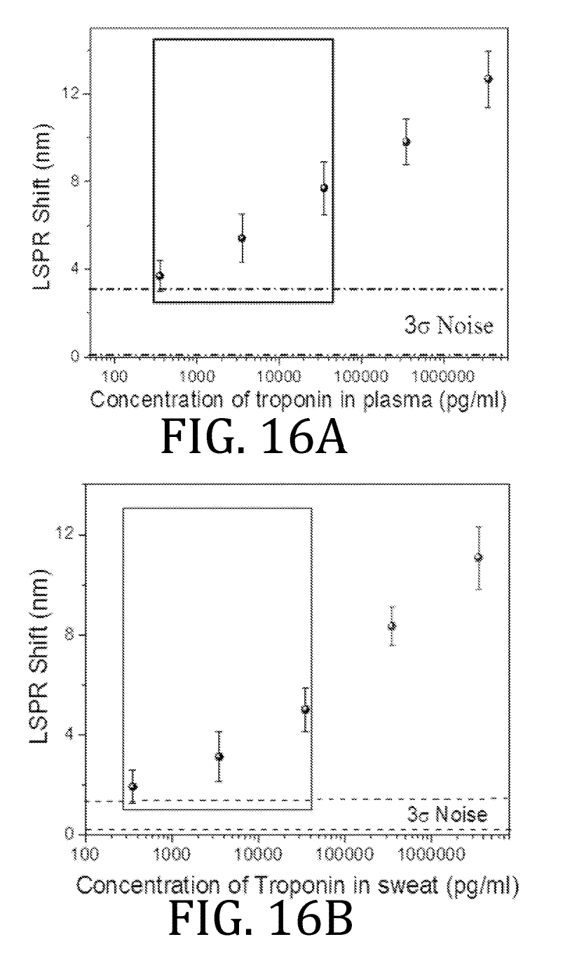

Peptide-based BPDs prepared in accordance with embodiments of the present invention were used in detection of troponin in complex physiological fluids, including, for example, human plasma and sweat. To reduce nonspecific binding, the BPDs were first exposed to 1% HAS to block non-specific sites. The measured LSPR shift exhibited a monotonic increase with increased troponin concentration in both human plasma and artificial eccrine sweat (FIG. 16A). The limit of detection of cTnI in human plasma was 353 pg/mL, which was within physiological relevant concentration range of troponin in human plasma (0.010 ng/mL to 10 ng/mL). Thus, the BPDs were shown to be an ultrasensitive detection system for cTnI. Moreover, detection of troponin in artificial eccrine sweat at physiologically-relevant concentration was also accomplished (see, FIG. 16B).

### Example 6

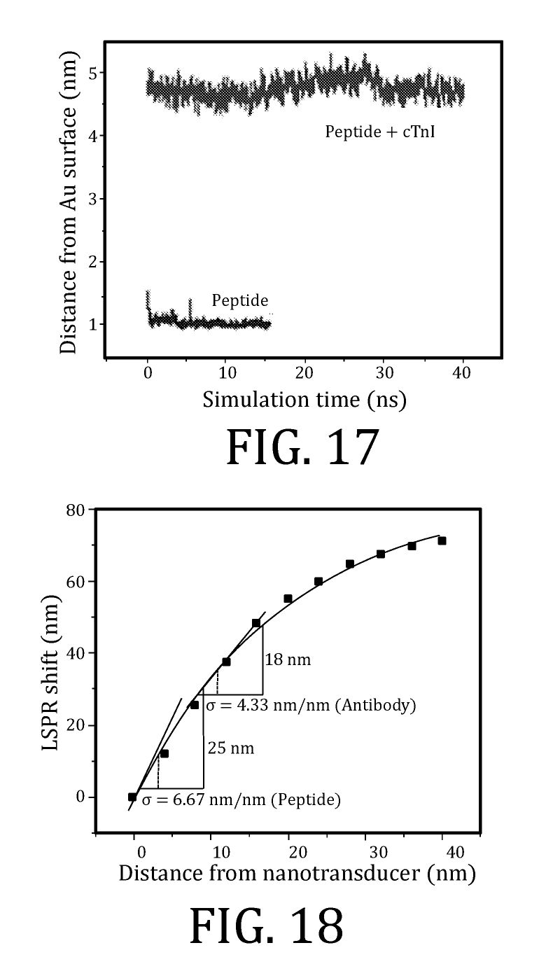

Atomic Force Microscopy (“AFM”) imaging revealed the thickness of the peptide layer (0.96 nm) on AuNR to be significantly smaller than that of the antibody (4.24 nm). The smaller thickness of the peptide layer renders higher sensitivity of BPDs using peptide recognition elements. For calculating the thickness layer from computer models (for example, NAMD, which was developed by the Theoretical and Computational Biophysics Group in the Beckman Institute for Advanced Science and Technology at the University of Illinois at Urbana-Champaign) the gold surface was aligned in the XY plane and the thickness of the peptide or peptide-cTnI complex on the gold surface is calculated as the maximum differences on the z-coordinates. FIG. 17 shows time evolution of the thickness of the peptide layer and the peptide-cTnI layer. Saturation values were 1.03±0.03 nm and 4.74±0.11 nm for the peptide and peptide-cTnI complex, respectively. AFM measurements of the peptide conjugated AuNR after cTnI binding revealed that the diameter of AuNRs increased by 3.8 nm. Adding the thickness of peptide in dry state (about 0.9 nm), the thickness of protein on the surface of the nanorod is about 4.8 nm, which is in excellent agreement with that predicted by the MD simulations.

### Example 7

Distance-dependent sensitivity of the AuNPs nanotransducers was probed by depositing polyelectrolyte multilayers using layer-by-layer (“LBL”) assembly. Extinction spectra were collected after the deposition of each bilayer comprised of poly(allylamine hydrochloride) (“PAH”) as a positively charged polymer and poly(styrene sulfonate) (“PSS”) as a negatively charged polymer. The LSPR wavelength shift exhibited a characteristic decay with increased distance from the surface of the nanorods (i.e., increasing number of polyelectrolyte layers).

By defining a distance-dependent refractive index sensitivity parameter (σ), which is the LSPR shift caused by deposition of 1 nm thick dielectric layer (polyelectrolyte multilayers in the present case) at a predetermined distance from the surface of the transducer. Such a parameter can be easily deduced at different distances from the AuNR surface, as shown in FIG. 18. The extinction measurements after troponin binding to the bioconjugated AuNR were performed in the dry state. So, considering that the thickness of peptide and antibody, respectively, the values of σ is found to be about 6.67 nm/nm, whereas in the case of antibody, σ was found to be 4.33 nm/nm.

From distance-dependent refractive index sensitivity measurements, it can be seen that the local refractive index sensitivity with peptide recognition elements is nearly 50% higher than compared to that with antibody-based recognition elements. A nearly 100% higher LSPR shift was observed with peptides as compared to antibodies as recognition elements.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the scope of the general inventive concept.

What is claimed is:

1. A biomarker sensor comprising:
a substrate;
a metallic layer on a surface of the substrate, the metallic layer being localized surface plasmon resonance reactive; and
a receptor layer on a surface of the metallic layer opposing the substrate, the receptor layer configured to selectively bind a biomarker and having a thickness less than about 15 nm.

2. The biomarker sensor of claim 1, wherein the receptor layer comprises a plurality of peptides, each peptide of the plurality having a number of amino acids ranging from 12 to 65.

3. The biomarker sensor of claim 1, wherein the metallic layer comprises a plurality of metallic nanoparticles.

4. The biomarker sensor of claim 3, wherein metallic nanoparticles of the plurality comprise silver, gold, or a combination thereof.

5. The biomarker sensor of claim 1, wherein the substrate is selected from a group consisting of glass, filter paper, and a polymer.

6. A biomarker sensing device comprising:
a substrate having a surface;
a first biomarker sensor on the surface of the substrate, the first biomarker sensor comprising;
a first metallic layer on the surface of the substrate, the metallic layer being localized surface plasmon resonance reactive; and
a first receptor layer on a surface of the first metallic layer opposing the substrate, the first receptor layer configured to selectively bind a first biomarker;
a second biomarker sensor on the surface of the substrate and spaced away from the first biomarker sensor, the second biomarker sensor comprising:
a second metallic layer on the surface of the substrate, the metallic layer being localized surface plasmon resonance reactive; and
a second receptor layer on a surface of the second metallic layer opposing the substrate, the second receptor layer configured to selectively not to bind the first biomarker; and
a flow path extending across the surface of the substrate, wherein a first portion of the flow path configured to intersect the first biomarker sensor and a second portion of the flow path configured to intersect the second biomarker sensor.

7. The biomarker sensor of claim 6, wherein the first receptor layer has a thickness that is less than 15 nm.

8. The biomarker sensor of claim 7, wherein the second receptor layer has a thickness that is less than 15 nm.

9. The biomarker sensor of claim 6, wherein the first receptor layer comprises a first plurality of peptides, each peptide of the first plurality having a number of amino acids ranging from 12 to 65.

10. The biomarker sensor of claim 9, wherein the second receptor layer comprises a second plurality of peptides, each peptide of the second plurality having a number of amino acids ranging from 12 to 65.

11. The biomarker sensor of claim 6, wherein the metallic layer comprises a plurality of metallic nanoparticles.

12. The biomarker sensor of claim 11, wherein metallic nanoparticles of the plurality comprise silver, gold, or a combination thereof.

13. The biomarker sensor of claim 6, wherein the substrate is selected from a group consisting of glass, filter paper, and a polymer.

14. The biomarker sensor of claim 6, wherein the second receptor layer is further configured to selectively bind a second biomarker.

15. The biomarker sensor of claim 6, wherein the flow path further comprises a third portion configured to intersect a third portion having a negative control group on the surface of the substrate.

16. The biomarker sensor of claim 6, further comprising:
a detector configured to measure a physical character of a first metallic layer, wherein the detector returns a change in the physical character when the first biomarker binds to the first receptor layer or, alternatively, the detector returns no change in the physical character when the first biomarker is not bound to the first receptor layer.

17. A biomarker sensor system comprising:
a substrate having a surface;
a metallic layer on the surface of the substrate, the metallic layer being localized surface plasmon resonance reactive;
a receptor layer on a surface of the metallic layer opposing the substrate, the receptor layer configured to selectively bind a biomarker;
a fluid path configured to intersect the receptor layer such that a specimen may flow across the surface of the substrate and the receptor layer; and
a detector configured to measure a physical character of the metallic layer,
wherein the detector returns a change in the physical character when the specimen comprises the desired biomarker after the desired biomarker binds to the receptor layer or, alternatively, the detector returns no change in the physical character when the specimen does not comprise the desired biomarker and no desired biomarker binds to the receptor layer.

18. The biomarker sensor system of claim 17, wherein the metallic layer comprises a plurality of metallic nanoparticles and the receptor layer comprises a plurality of peptides coupled to metallic nanoparticles of the plurality, each peptide of the plurality having a number of amino acids ranging from 12 to 65.

19. The biomarker sensor system of claim 17, wherein the physical character is reflected light.

20. The biomarker sensor system of claim 17, wherein the detector is a localized surface plasmon resonance spectrometer.


SEQUENCE LISTING (4 sequences)

SEQ ID NO: 1 - protein, 12 aa, Homo sapiens
  ref <301>: AUTHORS: JOHN PIL PARK et al.
  ref <302>: TITLE: High affinity peptides for the recognition of the heart disease biomarker troponin I identified using phage display
  ref <303>: JOURNAL: Biotechnology and Bioengineering
  ref <304>: VOLUME: 105
  ref <305>: ISSUE: 4
  ref <306>: PAGES: 678-686
  ref <307>: DATE: 2010-03-01
  ref <301>: AUTHORS: J WU et al.
  ref <302>: TITLE: Development of a troponin I biosensor using a peptide obtained through phage display
  ref <303>: JOURNAL: Analytical Chemistry
  ref <304>: VOLUME: 82
  ref <305>: ISSUE: 19
  ref <306>: PAGES: 8235-8243
  ref <307>: DATE: 2010-09-10
FYSHSFHENWPS

SEQ ID NO: 2 - protein, 13 aa, Artificial Sequence
  Artificial sequence derived from Seq. ID. No. 1.
FYSHSFHENWPSC

SEQ ID NO: 3 - protein, 13 aa, Homo sapiens
YHPNGMNPYTKAG

SEQ ID NO: 4 - protein, 14 aa, Artificial Sequence
  Artificial sequence derived from Seq. ID. No. 3.
YHPNGMNPYTKAGC